(19) United States
(12) Patent Application Publication
(10) Pub. No.: US 2007/0003644 A1
(43) Pub. Date: Jan. 4, 2007

(54) Concentrated polyphenolic product and process for making the same

(75) Inventors: Sarabjit S. Randhava, Evanston, IL (US); Sikander S. Randhava, Evanston, IL (US); Surjit S. Randhava, Evanston, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: UNITEL TECHNOLOGIES, INC., Mount Prospect, IL (US)

(21) Appl. No.: 11/405,376

(22) Filed: Apr. 17, 2006

Related U.S. Application Data
(60) Provisional application No. 60/671,792, filed on Apr. 16, 2005.

(51) Int. Cl. A61K 36/87

(52) U.S. Cl. 424/766

(57) ABSTRACT

A process for preparing a concentrated polyphenolic product is disclosed. The process generally includes the extraction of polyphenolic compounds from grape material using a solvent containing ethanol and water, and further includes steps of extract filtration and extract concentration to provide either a liquid polyphenolic concentrate or a powdered polyphenolic extract. The concentrated polyphenolic product may be used in food products and dietary supplements to afford the health benefits of consuming red wine. An additional process for preparing a concentrated ethanol product with a prefermentation step in addition to a concentrated polyphenolic product also is disclosed.

### CROSS-REFERENCE TO RELATED APPLICATIONS

The benefit under 35 U.S.C. § 119(e) of U.S. Provisional Patent Application Ser. No. 60/671,792 (the disclosure of which is hereby incorporated by reference), filed Apr. 16, 2005, is hereby claimed.

### BACKGROUND OF THE INVENTION

1. Field of the Disclosure

The disclosure relates to an integrated process for the extraction of substantially all soluble polyphenolic compounds from grape material and, optionally, the simultaneous production of a grape-based ethanol product. More specifically, the extracted polyphenolic material may be produced in a liquid concentrate form or in a dry powdered form.

2. Brief Description of Related Technology

Polyphenolic compounds extracted from grape material are believed to provide cardio-protective and other health benefits of red wine, without the alcohol. This health benefit is commonly referred to as the “French Paradox.” Consumption of the extracted compounds provides the observed cellular life extension associated with resveratrol consumption. While the precise reason for the observed health benefits is not currently known, the following paragraphs describe some of the benefits and hypotheses.

Scientific evidence supports the contention that two glasses of red wine a day can produce significant health benefits, including, for example the prevention of neurodegenerative diseases, such as Alzheimer's and Parkinson's diseases, the prevention of the cardiovascular disease atherosclerosis (the collection of plaque in the arteries and a leading cause of heart disease), the prevention of food poisoning, the prevention of dysentery, and a reduction in human mortality rates. The most widely accepted theory is based on the approximately 400 polyphenolic substances in red wine, which are collectively believed to provide health benefits due to their free-radical scavenging, antioxidant activity. See Frankel et al., Inhibition of human LDL oxidation by resveratrol, THE LANCET, vol. 341, pp. 1103-1104 (1993).

Recent work shows that the compound resveratrol (found in red wine) activates the same corporal response as severe calorie restriction, blocking the uptake of a class of enzymes involved in cellular death (sirtuins), thereby increasing the lifespan of the cell. Findings show that organisms subjected to severe calorie restriction have life spans approximately 70% greater than conventionally fed counterparts. Organisms fed the red wine compound resveratrol achieved the identical increase in longevity. Furthermore, organisms subjected to a starvation diet and resveratrol achieved the same 70% increase in lifespan, suggesting that these effects follow identical pathways and cannot be magnified by inclusion of both stimuli. See Konrad et al., Small Molecule Activators of Sirtuins Extend Saccharomyces Cerevisiae Lifespan, NATURE, vol. 425, pp. 191-196 (2003).

While excessive consumption of saturated fats and cholesterol-laden foods are linked to increased mortality from coronary heart disease and atherosclerosis, French dietary habits include both of these perilous traits, and yet the French suffer from an unusually low incidence of coronary problems (the “French Paradox”). The annual per capita wine consumption by French adults is 60 liters, compared to 7 liters in the United Sates, and this high rate of red wine consumption has been suggested as a possible contributing factor to the relatively low incidence of cardiovascular disease in that country. See Kinsella et al., Wine and Health: The possible role of phenolics, flavonoids and other antioxidants. Potential Health Effects of Components of Plant Foods and Beverages in the Diet, University of California Davis, Proceedings, August 14-15 pp. 107-121 (1992). Specifically, while adults in France, the United Kingdom, and the United States have roughly equal plasma cholesterol levels (i.e., about 215-240 mg/dl), the English coronary heart mortality rate was almost four times the French rate, the American rate was about twice the French rate, and the effect is even more pronounced when local variations in French red wine consumption are considered. See Renaud and de Lorgeril, Wine, alcohol, platelets and the French Paradox for coronary heart disease, THE LANCET, vol. 339, pp. 1523-1526 (1992).

Also of great interest to the medical community is the ability of these polyphenolic compounds to raise the levels of high-density lipoproteins (HDL, or “good” cholesterol) in the blood while decreasing the low-density lipoproteins (LDL, or “bad” cholesterol). See Frankel et al., Principal phenolic phytochemicals in selected California wines and their antioxidant activity in inhibiting oxidation of human low-density lipoproteins, J. AGRIC. FOOD CHEM., vol. 43, pp. 890-894 (1995). For example, in vitro studies indicte that wine polyphenolics are twice as effective as alpha-tocopherol in inhibiting LDL oxidation, and that these polyphenolic antioxidants were found in all tested grape varieties and wines. See Kanner et al, Natural antioxidants in grape and wine, J. AGRIC. FOOD CHEM., vol. 42, pp. 64-69 (1995). Similarly, studies demonstrate the lipoprotein-bound activity of phenols (and in particular flavonoids), thus suggesting that phenols can bind to LDL in vivo (as do tocopherols) before exerting their beneficial effect as antioxidants. See Vinson et al., Plant polyphenols exhibit lipoprotein-bound antioxidant activity using in vitro oxidation model for heart disease, J. AGRIC. FOOD CHEM., vol. 43, pp. 2798-2799 (1995).

Resveratrol is found in the skin of grapes, and has been the subject of medical research because of its high antioxidant properties, anti-inflammatory effects, and recent demonstrations of its anti-carcinogenic properties. See Jang et al., Cancer Chemopreventive Activity of Resveratrol, a Natural Product Derived from Grapes, SCIENCE, vol. 10, pp. 218-221 (1997).

However, something other than antioxidants in red wine may reduce coronary heart disease. The concentration of red wine polyphenolics is directly related to the level of inhibition in the synthesis of endothelin-1 (ET-1), a peptide crucial in the development of coronary atherosclerosis. This suggests that it is unlikely that antioxidant activity alone is responsible for blocking ET-1 synthesis. See Corder et al., Endothelin-1 Synthesis Reduced by Red Wine, NATURE, vol. 414, pp. 863-864 (2001).

The association between red wine and health benefits is not due to the color of the grape, but rather the process in red winemaking by which the whole fruit, particularly the grape skin, is allowed to ferment with the wine, allowing skin, seed, and flesh components to solubilize into the wine. Studies demonstrate that the daily consumption of red wine for two weeks reduces the susceptibility of plasma LDL to undergo lipid peroxidation, while consumption of white wine shows the opposite effect, suggesting that it is the polyphenolics of red wines (i.e., and not the ethanol) which inhibit LDL oxidation. See Fuhrman et al., Consumption of red wine with meals reduces the susceptibility of human plasma and low density lipoprotein to lipid peroxidation, AM. J. CLIN. NUTR., vol. 61, pp. 549-554 (1995).

Generally, the prior art discloses methods for the selective extraction and recovery of certain compounds from plant material generally, and grape material more specifically. However, it does not sufficiently teach or suggest to one of ordinary skill in the art how to recover substantially all of the polyphenolics from grape material in substantially the same relative amounts as found in red wine. For example:

U.S. Pat. No. 3,436,407 discloses a method for the extraction of hydroxyflavans and the administration of these compounds to humans for health purposes. A selective extraction process for removing the hydroxyflavan 3,4-diols having a lower degree of polymerization (in particular monomers, dimers, trimers, tetramers, and pentamers) is disclosed.

U.S. Pat. No. 5,912,363 discloses a hot water extraction method for the extraction of proanthocyanidins from plant material, including grape seeds. The proanthocyanidins are recovered from the water extract by elution from a chromatographic column with a polar solvent.

U.S. Pat. No. 5,968,517 discloses a hot water extraction method for the extraction and isolation of proanthocyanidins from biological material, including tree bark, grape seeds, and grape skins. The process includes the steps of comminuting with deoxygenated water, separating the solids from the liquor, concentrating the liquor, and drying the extracted product.

U.S. Pat. No. 6,544,581 discloses a hot water extraction method for the extraction and enrichment of polyphenolics from whole grapes, grape seeds, and grape pomace. A dual pH treatment of the aqueous extract, followed by recovery of the polyphenolics by chromatographic elution with a polar solvent is also disclosed. The extraction process is designed to maximize the extraction of procyanidins having a low degree of polymerization, while simultaneously minimize the extraction of procyanidins having a high degree of polymerization.

### SUMMARY OF THE INVENTION

One aspect of the disclosure provides a process for preparing a concentrated polyphenolic product. The process generally includes extracting substantially all polyphenolics from grape material with a solvent at conditions suitable to form a liquid polyphenolic extract, the solvent consisting essentially of water and ethanol. The process also includes removing the solvent from the liquid polyphenolic extract to form a liquid polyphenolic concentrate.

Another aspect of the disclosure provides an extraction process comprising extracting polyphenolics from grape pomace with a solvent to form a liquid polyphenolic concentrate, the solvent consisting essentially of water and ethanol.

Other aspects of the disclosure include liquid polyphenolic concentrates, powdered polyphenolic extracts, and concentrated ethanol products produced by the above processes.

Additional features of the invention may become apparent to those skilled in the art from a review of the following detailed description, taken in conjunction with the drawings, the examples, and the appended claims.

### BRIEF DESCRIPTION OF THE DRAWING FIGURES

For a more complete understanding of the disclosure, reference should be made to the following detailed description and accompanying drawing wherein:

FIG. 1 is a schematic view of a process flow diagram for one embodiment of the disclosed extraction process; and,

FIG. 2 is a schematic view of a process flow diagram for another embodiment of the disclosed extraction process.

While the disclosed processes and products made therefrom are susceptible of embodiments in various forms, there are illustrated in the drawings (and will hereafter be described) specific embodiments of the invention, with the understanding that the disclosure is intended to be illustrative, and is not intended to limit the invention to the specific embodiments described and illustrated herein.

### DETAILED DESCRIPTION OF THE INVENTION

The invention generally relates to processes for preparing a concentrated polyphenolic product. Unless specified otherwise, concentrations of components disclosed herein are given on weight basis relative to the total weight of the components (wt. %). For example, the concentration of ethanol in an ethanol-water solvent is based on the combined weight of ethanol and water only. However, the concentration of a component in a solid-liquid slurry is based on the combined weight of the solids (e.g., solid grape material) and liquid (e.g., liquid grape material, the ethanol portion of the solvent, and the water portion of the solvent). Temperatures and pressures are given standard designations. Unless defined otherwise, all other technical and scientific terms used herein have the meaning commonly understood by a person skilled in the art to which this disclosure belongs.

One aspect of the disclosure provides a process for preparing a concentrated polyphenolic product. The process generally includes extracting substantially all polyphenolics from grape material with a solvent at conditions suitable to form a liquid polyphenolic extract, the solvent consisting essentially of water and ethanol. The process also includes removing the solvent from the liquid polyphenolic extract to form a liquid polyphenolic concentrate. Another aspect of the disclosure provides an extraction process comprising extracting polyphenolics from grape pomace with a solvent to form a liquid polyphenolic concentrate, the solvent consisting essentially of water and ethanol.

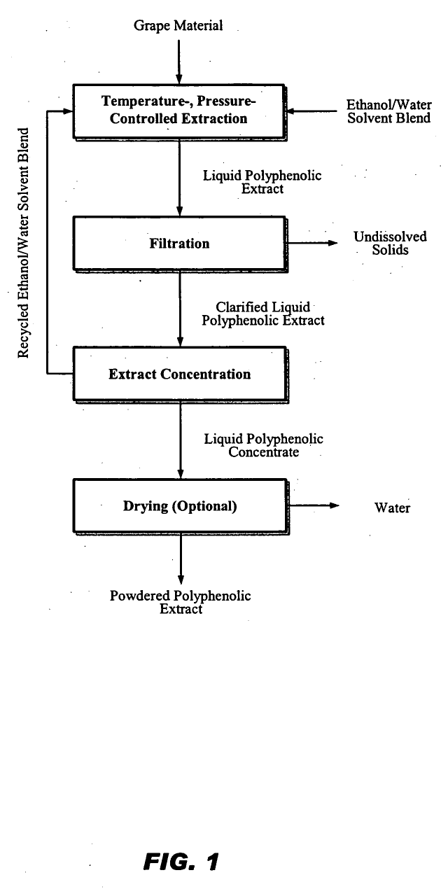

With reference to FIG. 1 and the following process materials and steps, preferred embodiments of the aforementioned process are described.

Grape Material

The starting grape material for the disclosed process generally includes the polyphenolic-containing portion of grapes such as, for example, grape pomace, grape seeds, whole grape clusters, grape fermentation products, and combinations thereof.

A preferred form of grape material includes grape pomace, which is the solid remains from the grape pressing step used to produce certain types of champagne, white wines, and/or grape juice. Grape pomace has not been fermented, or been allowed to steep in wine-fermenting tanks and, thus, has not lost its polyphenolic content (which includes all of the polyphenolic compounds present in the raw grape) by extraction into the champagne or white wine product. A preferred form of grape pomace is that from red grapes, and a more preferable form of grape pomace is that from Pinot Noir grapes.

When the grape material is grape pomace or whole grape clusters, it is preferably in a crushed form. Crushing bruises the grape seeds and increases the contact area between the solvent and solid grape material such as seeds, skin, etc. In one embodiment, the grape pomace is the crushed material retrieved from the presses of certain types of wine- or champagne-making operations. In another embodiment, the whole grape clusters are crushed in a standard grape crusher, and are introduced into a fermentation tank along with the juice, where the material ferments and produces the ethanol which will later serve as an extraction agent.

The grape material may be used immediately when formed, or may be stored for later use. The grape material is preferably stored in the absence of light and at sub-freezing temperatures. When frozen, the grape material may be first defrosted in a separate process step, or it may be fed directly into the extraction vessel with the extraction solvent, and defrosted by the application of heat in the extraction vessel. If the material is defrosted in the extraction vessel, the total residence time for the extraction step should be increased accordingly by the time required to first thaw the frozen grape material.

Prior to extraction, the grape material may be optionally treated to increase the accessibility of the solvent to the desired polyphenolic compounds. Optional treatment steps include a comminution treatment and an enzymatic treatment, which may be used either in conjunction or in isolation.

The comminution treatment includes processes that break down or rupture plant cell walls or tissues, including methods known in the art such as milling, grinding, or other suitable operations that reduce the material to smaller particles.

Enzymatic treatment also breaks down the plant cell walls. Such treatments can include exposure of the grape material to enzymes such as, for example, cellulases, hemicellulases, pectinases, proteinases, or combinations thereof. The enzymatic treatment is performed using established protocols for the selected enzymes.

Extraction Solvent

The extraction solvent for the disclosed process consists essentially of ethanol and water. Additional solvent contents beyond ethanol and water, if any, should not affect the ability of the solvent to remove substantially all of the polyphenolics from the grape material. Preferably, additional solvent contents will be safely ingestible by humans and/or animals at the levels used (and at the levels remaining in a final product).

The ethanol-water solvent mixture advantageously contains about 5 wt. % to about 90 wt. % ethanol. The solvent mixture preferably contains at least about 12 wt. % ethanol, more preferably at least about 25 wt. % ethanol, and most preferably at least about 50 wt. % ethanol. The solvent mixture preferably contains not more than about 80 wt. % ethanol, more preferably not more than about 70 wt. % ethanol, and most preferably not more than about 60 wt. % ethanol.

The selection of a particular ethanol weight concentration depends upon parameters such as the ratio of polyphenolic compounds desired from the feedstock (because various polyphenolic compounds have differing solubilities in ethanol as compared to water), the energy requirements for evaporation or distillation of the solvent to concentrate the desired compounds prior to final drying, and the costs/benefits associated with regulations and restrictions affecting the use of ethanol.

Prior to extraction, grape material is mixed with an amount of extraction solvent to form a solid-liquid slurry. The weight ratio of grape material:solvent is preferably about 1:4 to about 4:1, more preferably about 1:2 to about 2:1, and most preferably about 1:1.

In one embodiment, grape pomace having a solids content of about 50 wt. % and a water content of about 50 wt. % is mixed with a solvent containing about 50 wt. % water and 50 wt. % ethanol to create a solid-liquid slurry containing about 25 wt. % solids, about 25 wt. % ethanol, and about 50 wt. % water, based on the total weight of solid-liquid slurry.

Extraction of Polyphenolics and Other Materials

Once formed, the solid-liquid slurry of grape material and solvent is fed into a closed extraction vessel and processed at conditions suitable to form a liquid polyphenolic extract in which substantially all of the polyphenolics have been recovered from the grape material into the extract.

The extraction of the polyphenolic and other dissolved compounds from the grape material is carried out at a temperature preferably of at least about 30° C., and more preferably in the range of about 90° C. to about 120° C.

The extraction of the polyphenolic and other dissolved compounds from the grape material may be carried out at ambient pressure, but is preferably carried out at a pressure in the range of about 15 psig to about 40 psig.

The extraction of the polyphenolic and other dissolved compounds from the grape material is carried out with an extraction residence time preferably in a range of about 20 minutes to about 120 minutes, and more preferably about 30 minutes to about 90 minutes.

In a preferred embodiment, the extraction temperature is about 110° C., the extraction pressure is about 25 psig, and the extraction time is about 60 minutes.

Preferably, the extraction is performed in a single stage, batch extraction step, although multiple batch extraction units may be employed in parallel to increase processing capacity. However, continuous solid-liquid extractors or multiple batch extractors in series also may be used. In these cases, the relevant amount of solvent for determining the weight ratio of grape material:solvent is the total amount of solvent fed to an extractor either over time (as in the case of continuous solid-liquid extractors) or over a series of stages (as in the case of batch solid-liquid extractors in series).

The disclosed extraction step is capable of removing substantially all of the polyphenolic compounds contained in the grape material. Polyphenolic compounds generally include organic compounds having multiple phenolic functional groups on a single molecule, regardless of whether the —OH groups are present on a single aromatic ring or distributed among multiple aromatic rings. Polyphenolic compounds may be further classified as flavonoids and non-flavonoids. Examples of non-flavonoids contained in grape material include, for instance, resveratrol and gallic acid. Examples of flavonoids contained in grape material include, for instance, quercitin, anthocyanin, and catechin (and its derivatives, such as epicatechin and epicatechin gallate).

The various monomeric polyphenolic compounds are also naturally present in the grape material in oligomeric and polymeric forms known as proanthocyanins. The oligomeric polyphenolics have a degree of polymerization ranging from 2 to 7, and the polymeric polyphenolics have a degree of polymerization ranging from 8 to about 16. The disclosed extraction step is capable of removing substantially all of the monomeric, oligomeric, and polymeric polyphenolic compounds

Because of the wide variation in the chemical properties of the targeted polyphenolic compounds, in particular with respect to their molecular weight and degree of —OH substitution, there is a correspondingly wide variation in the solubility characteristics of the individual polyphenolic species.

One aspect of the process is the disclosure of a solvent (i.e., the ethanol-water blend described above) capable of extracting substantially the entire complex of (i.e., substantially all) polyphenolics contained or present in the grape material, preferably in a single extraction stage. Thus, a liquid polyphenolic concentrate or a powdered polyphenolic extract formed by the disclosed process contains substantially all of the polyphenolic compounds originally contained in the grape material, including the various monomeric, oligomeric, and polymeric species. Another aspect of the present process is its ability to recover various monomeric, oligomeric, and polymeric species from the grape material into a liquid polyphenolic concentrate or into a powdered polyphenolic extract in substantially the same relative amounts as originally contained in the grape material.

The disclosed extraction step also is capable of removing substantially all of other dissolved materials contained in the grape material, including, for example, polysaccharides and non-polyphenolic organic acids.

Removal of Solids from the Extract

Following the extraction step, the liquid polyphenolic extract containing the solvent and polyphenolic compounds also contains undissolved solid material. The undissolved solid material may be separated from the liquid polyphenolic extract via filtration. This separation can be performed using methods and materials known in the art. Preferably, however, filtration is performed via membrane plate and frame filtration, centrifugation, any other filter aid suitable for the production of a product safe for human consumption, and combinations thereof.

In an embodiment, the liquid polyphenolic extract discharged from the extraction vessel is fed to a membrane plate and frame filtration system to yield a pressed, clarified liquid polyphenolic extract having about 7.5 wt. % dissolved solids.

Concentration of the Extract

The liquid polyphenolic extract is further processed to remove solvent from the extract to form a liquid polyphenolic concentrate using methods known in the art such as, for example, evaporation and/or distillation. Preferably, the liquid polyphenolic concentrate is substantially free of ethanol. Additionally, following its removal from the liquid polyphenolic extract, the solvent is preferably recycled to the extraction step.

In an embodiment, the liquid polyphenolic extract is concentrated using a falling film evaporator operating at a temperature in a range of about 25° C. to about 98° C., and at an absolute pressure of about 0.25 atm. The resulting liquid polyphenolic concentrate has a syrup-like consistency, contains about 40 wt. % dissolved solids, and contains substantially all of the dissolved polyphenolic compounds, organic acids, and polysaccharides that were originally present in the grape material.

The concentrated polyphenolic extract may be used as a liquid polyphenolic concentrate, or it may be subjected to an additional drying step to form a powdered polyphenolic extract. The drying step may use methods known in the art such as, for example, lypholization (i.e., freeze drying), spray drying, or refractance window drying. In an embodiment, refractance window drying is used to form a reddish purple powdered polyphenolic extract containing a high concentration of the polyphenolic compounds, polysaccharides, and other non-flavonoid compounds found in red wine.

Preparation of an Ethanol Product

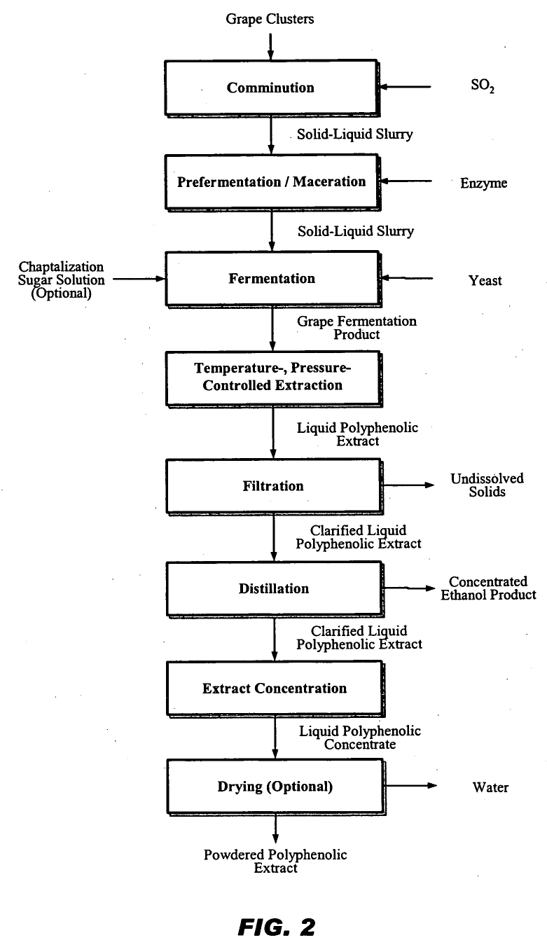

In another embodiment, grape clusters serve as the feedstock for the process, which process is illustrated schematically in FIG. 2. The grape clusters are crushed, and the resulting grape juice and grape pomace are then fermented together to produce a grape fermentation product containing ethanol (from the fermentation process) and water (from the grapes themselves), which two liquids serve as the solvent mixture for a subsequent extraction step.

The fermentation step applied to the grape clusters is performed according to standard steps known in the art of fermenting alcoholic products from plant material. In an illustrative embodiment, the crushed grape clusters are added to an agitated prefermenter/macerator. Sulfur dioxide (SO₂) is also added to the slurry to suppress the growth of native yeasts and bacteria, and to bind readily with-the anthocyanin pigments in freshly crushed grape clusters, thus making them more soluble. A pectinase enzyme also is added to the slurry to macerate the grape clusters, thereby breaking down cell walls and making the desired polyphenolic compounds more accessible to the extraction solvents. The slurry from the prefermenter-macerator is then pumped to a closed rotary fermenter, into which cultured yeast also is added to rapidly induce fermentation. The slurry is generally fermented at a temperature of about 18° C. to about 35° C. A higher fermentation temperature leads to a fast, vigorous fermentation, with the resulting grape fermentation product (e.g., wine) having more complexity and vinosity. The total fermentation time is about 3 days to 10 days, depending on the time required to achieve an ethanol content of about 12 vol. % to 15 vol. % in the grape fermentation product.

Optionally, a chaptalization step may be added to the fermentation process. Chaptalization is a process practiced when the grape material has a low natural sugar content, and involves the addition of cane or beet sugar prior to fermentation to increase the final ethanol content of the grape fermentation product. The addition of about 17 g/L to about 19 g/L of sugar to the slurry results in an increase of about 1 wt. % ethanol in the grape fermentation product. Chaptalization preferably is performed at a level appropriate to yield a grape fermentation product containing 25 wt. % ethanol, based on the total weight of ethanol, water, and solid grape material.

After formation of the grape fermentation product, the grape fermentation product is fed to the closed extraction vessel described above. Optionally, additional ethanol and/or water solvent can be added to the extraction vessel to adjust the solvent ethanol concentration based on the considerations discussed above related to the extraction solvent.

The preferred extraction conditions with respect to temperature, pressure, and residence time are the same as those discussed above. Once the extraction step is complete, a liquid polyphenolic extract is formed, and the liquid polyphenolic extract is further filtered as described above. The final, clarified liquid polyphenolic extract has about 7.5 wt. % dissolved solids.

In this other embodiment, the ethanol and excess water in the liquid polyphenolic extract are not recycled for use in a subsequent extraction process. Rather, portions of ethanol, water, and the wine top notes in the liquid polyphenolic extract are recovered in the overhead stream of a vacuum distillation step. For example, a continuous mode vacuum distillation step with an inlet flow rate of about 500 kg/hr (containing 14 wt. % ethanol) and a desired overhead concentration about 75 vol. % (i.e., 150 proof) ethanol requires approximately 4 equilibrium mass transfer stages and approximately 150 kg/hr of steam. The distillation appratus is fabricated from copper to prevent catalytic degradation of the wine top notes, which could otherwise be lost if contacted with steel or stainless steel.

The vacuum distillation overhead stream is a concentrated ethanol product. The concentrated ethanol product can be diluted to about 40 vol. % ethanol immediately to yield a marketable commodity commonly referred to as eau-de-vin brandy. Alternatively, the concentrated ethanol product may be further aged in brandy manufacturing processes.

The vacuum distillation bottoms stream, which is substantially free of ethanol but still contains water and substantially all of the polyphenolics in the original grape material is subjected to the extract concentration steps described above. Specifically, a liquid polyphenolic concentrate can be formed and, with the inclusion of an additional drying step, so too can a powdered polyphenolic extract be formed.

Polyphenolic Products

While the health benefits of red wine have been well documented, medical research remains inconclusive about the relative effect of each compound present in red wine. The variety of health benefits gained from red wine consumption might come from multiple actions of compounds working synergistically. Products made according to the processes disclosed herein offer the health benefits of red wine because they contain the full spectrum of compounds found in red wine, replicating the level and ratio of polyphenolic compounds found in red wine, thus accurately duplicating the health benefits of red wine.

The concentrated polyphenolic products obtained by the processes disclosed herein are suitable for consumption by humans and other animals to obtain the health benefits therefrom. Specifically, either the liquid polyphenolic concentrate or the powdered polyphenolic extract may be used in dietary supplements, in nutraceuticals, or as additives for inclusion in functional foods (i.e., processed food products that have been fortified with additional health-related components such as vitamins or, in this case, polyphenolic compounds).

The health benefits of red wine consumption can be integrated into various food products using either the liquid polyphenolic concentrate or the powdered polyphenolic extract. For example, the polyphenolic-rich products can be formulated into food products as a nutritional fortifying agent, with the final blend in each case being itself a subject of the disclosure. Examples of such food products include, but are not limited to, alcoholic beverages, fruit juices, sport and energy drinks, bottled waters (flavored and unflavored), powdered beverages, concentrated beverages, geriatric beverages and shakes, health and sport bars, and yogurts.

Animal food products also may be fortified by the addition of the liquid polyphenolic concentrate or the powdered polyphenolic. Examples of such animal food products include, but are not limited to, dry and wet dog foods, dry and wet cat foods, and horse/large animal feeds.

The products obtained by the processes disclosed herein allow people who do not drink alcohol (either in general or in a particular situation) to take advantage of the health benefits of red wine by consuming polyphenolic-rich products that are substantially free of ethanol:

Another advantage of the processes disclosed herein is that the process feed material can comprise waste by-products obtained from the production other products, such as wine and champagne. For example, grape pomace recovered from the pressing of Pinot Noir grapes used to produce champagne yields a significant amount of all polyphenolic compounds present in the grape prior to pressing. Such a feedstock provides access to all of the polyphenolic compounds available in red wine, yet does not require the purchase of raw Pinot Noir grapes.

Throughout the specification, where compositions are described as including components or materials, it is contemplated that the compositions can also consist essentially of, or consist of, any combination of the recited components or materials, unless described otherwise. Combinations of components are contemplated to include homogeneous and/or heterogeneous mixtures, as would be understood by a person of ordinary skill in the art in view of the foregoing disclosure.

The foregoing description is given for clearness of understanding only, and no unnecessary limitations should be understood therefrom, as modifications within the scope of the invention may be apparent to those having ordinary skill in the art.

What is claimed is:

1. A process for preparing a concentrated polyphenolic product, the process comprising the steps of:
(a) extracting substantially all polyphenolics from grape material with a solvent at conditions suitable to form a liquid polyphenolic extract, the solvent consisting essentially of water and ethanol; and
(b) removing the solvent from the liquid polyphenolic extract to form a liquid polyphenolic concentrate.

2. The process of claim 1, wherein the ethanol is present in the solvent in a range of about 5 wt. % to about 90 wt. %, based on the total weight of the solvent.

3. The process of claim 1, wherein the conditions comprise a temperature of about 90° C. to about 120° C.

4. The process of claim 1, wherein the conditions comprise a pressure of about 15 psig to about 40 psig.

5. The process of claim 1, wherein the conditions comprise an extraction time of about 30 minutes to about 90 minutes.

6. The process of claim 1, wherein the weight ratio of grape material:solvent in the extracting step (a) is about 1:4 to about 4:1.

7. The process of claim 1, wherein the liquid polyphenolic concentrate is substantially free of ethanol.

8. The process of claim 1, wherein the grape material is selected from the group consisting essentially of grape pomace, grape seeds, grape clusters, a grape fermentation product, and combinations thereof.

9. The process of claim 8, wherein the grape material comprises grape pomace.

10. The process of claim 8, wherein the grape material comprises a grape fermentation product.

11. The process of claim 10, further comprising, prior to the removing step (b), distilling the liquid polyphenolic extract to form a concentrated ethanol product.

12. The process of claim 1, further comprising drying the liquid polyphenolic concentrate to form a powdered polyphenolic extract.

13. The process of claim 1, further comprising, prior to the removing step (b), filtering the liquid polyphenolic extract to remove undissolved solid material.

14. The process of claim 13, wherein the filtering comprises membrane plate and frame filtration.

15. The process of claim 1, further comprising comminuting the grape material either prior to or during the extracting step (a).

16. The process of claim 1, further comprising enzymatically treating the grape material either prior to or during the extracting step (a).

17. The process of claim 1, further comprising recycling the solvent recovered from the removing step (b) to the extraction step (a).

18. The liquid polyphenolic concentrate produced by the process of claim 1.

19. The powdered polyphenolic extract produced by the process of claim 12.

20. The concentrated ethanol product produced by the process of claim 11.

21. An extraction process comprising extracting polyphenolics from grape pomace with a solvent to form a liquid polyphenolic concentrate, the solvent consisting essentially of water and ethanol.

22. The extraction process of claim 21, further comprising drying the liquid polyphenolic concentrate to form a powdered polyphenolic extract.

23. The liquid polyphenolic concentrate produced by the process of claim 21.

24. The powdered polyphenolic extract produced by the process of claim 22.
